(19) United States
(12) Patent Application Publication
(10) Pub. No.: US 2010/0197983 A1
(43) Pub. Date: Aug. 5, 2010

(54) METHOD FOR THE PRODUCTION OF UNBRANCHED ACYCLIC OCTACTRIENES

(75) Inventors: Matthias Beller, Nienhagen (DE); Ralf Jackstell, Cuxhaven (DE); Surendra Harkal, Dortmund (DE); Dagmara Ortmann, Brig-Glis (CH); Franz Nierlich, Marl (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: OXENO OLEFINCHEMIE GMBH, Marl (DE)

(21) Appl. No.: 11/721,978

(22) PCT Filed: Oct. 20, 2005
(86) PCT No.: PCT/EP2005/055419

§ 371 Date: Dec. 29, 2009

(30) Foreign Application Priority Data
Dec. 16, 2004 (DE) 10 2004 060 520.3

(51) Int. Cl. C07C 11/21; B01J 31/02

(52) U.S. Cl. 585/16; 502/167; 585/538

(57) ABSTRACT

The invention relates to the preparation of one or more unbranched acyclic octatriene(s) by dimerization of 1,3-butadiene in the presence of a catalyst comprising a carbene ligand and an element of transition group VIII of the Periodic Table of the Elements as metal.

The invention relates to the preparation of one or more unbranched acyclic octatriene(s) by dimerization of 1,3-butadiene and a catalyst for this purpose.

Acyclic unbranched octatreienes are comonomers for the preparation of modified polyethylene or polypropylene. They can be utilized as component for the preparation of terpolymers, i.e. elastomers which are obtained by polymerization of monoolefins and a hydrocarbon having at least two double bonds in a molar ratio of 2:1. In addition, the unbranched acyclic octatrienes can be converted into the corresponding monoepoxides, diepoxides or triepoxides. These epoxides can, for example, be precursors for polyethers.

Linear octatrienes can be prepared, for example, by dimerization of 1,3-butadiene. In the dimerization of 1,3-butadiene, it is possible for higher oligomers, cyclic dimers such as 1,5-cyclooctadiene and vinylcyclohex-4-ene, branched and linear acyclic dimers and also mixtures thereof to be formed. Catalysts used for the dimerization of 1,3-butadiene to form linear dimers are usually complexes of the metals of transition group eight of the Periodic Table, for example those of iron, cobalt, rhodium, nickel or palladium.

Nickel complexes having a trivalent phosphorus compound as ligand are used as catalysts in, for example, DD 107 894, DD 102 688 and U.S. Pat. No. 3,435,088. In U.S. Pat. No. 4,593,140, a nickel-phosphinite complex in which the OR radical contains an amino group is used as catalyst.

A palladium-phosphine complex is used as catalyst in U.S. Pat. No. 3,691,249 and DD 102 376, a palladium-phosphite complex is used as catalyst in U.S. Pat. No. 3,714,284 and the bis(triphenylphosphine)palladium-maleic anhydride complex is used as catalyst in DE 16 68 326.

The abovementioned processes for the dimerization of 1,3-butadiene give linear octatrienes in sometimes very high yields, but they all have the disadvantage that the catalyst stability and/or activity is too low and/or the specific catalyst costs are too high for an economical industrial process. In addition, the phosphorus-containing ligands have the disadvantage that they are very sensitive to oxidation.

It is therefore an object of the invention to provide an alternative catalyst system which does not have one or more of the disadvantages of the catalyst systems of the prior art.

It has now surprisingly been found that 1,3-butadiene can be converted with very high selectivity and in a high space-time yield into linear octatrienes, virtually exclusively 1,3,7-octatriene, in the presence of a secondary alcohol and a base when a complex of a metal of transition group eight of the Periodic Table of the Elements having at least one carbene of the structural formula L, in particular 1,3-bis(2,6-diisopropylphenyl)-4,5-dimethyl-2-dehydro-3-hydroimidazole, as ligand is used as catalyst. This finding was not to be expected since, for example in DE 101 49 348, a palladium-carbene complex whose carbene unit is likewise a 1,3,4,5-tetrasubstituted 2-dehydro-3-hydroimidazole is, inter alia, used as catalyst for the telomerization of butadiene with an alcohol to form 1-alkoxy-2,7-octadiene.

The invention accordingly provides a process for preparing linear octatrienes from 1,3-butadiene or 1,3-butadiene-containing hydrocarbon mixtures, wherein the dimerization of the 1,3-butadiene is carried out in the presence of a secondary alcohol and a base, and a complex of a metal of transition group eight of the Periodic Table of the Elements having at least one carbene of the structure L

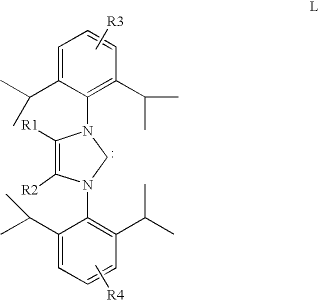

where R1 and R2=C₁-C₃-alkyl radical and R3 and R4=H or a C₁-C₃-alkyl radical, with the radicals R1 and R2 or R3 and R4 being able to be identical or different, as ligand is used as catalyst. The radicals R3 and R4 can be joined to the benzene ring in the 3, 4 or 5 position. The radicals R3 and R4 are preferably bound to the benzene ring in the 4 position.

The present invention likewise provides a mixture of octatrienes prepared by the process of a the invention and also provides for the use of such mixtures for preparing linear octenes.

The present invention also provides a carbene complex catalyst which is a ligand complex of a metal of transition group eight of the Periodic Table of the Elements which has at least one carbene of the structure L,

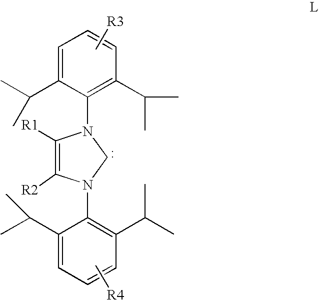

where R1 and R2=C₁-C₃-alkyl radical and R3 and R4=H or a C₁-C₃-alkyl radical, with the radicals R1 and R2 or R3 and R4 being able to be identical or different, as ligand.

The advantages of the process of the invention are that, owing to the significant differences in the boiling points, the reaction mixture can easily be separated into octatriene, starting material, secondary alcohols, by-products and catalyst and the catalyst which has been separated off can mostly be recirculated to the process. This results in an inexpensive process because both the separation costs and the catalyst costs are low.

In addition, the precursors of the ligands used according to the invention can be stored without problems for a relatively long time and the ligands are less oxidation-sensitive. The process of the invention has the further advantage that conversions of butadiene of above 80% are obtained and the yield of 1,3,7-octatriene is above 75%.

The process of the invention is described by way of example below without the invention, whose scope is defined by the claims and the description, being restricted thereto. The claims themselves are also part of the disclosure of the present invention. If ranges or preferred ranges are given in the following text, all theoretically possible subranges and individual values within these ranges are also part of the disclosure of the present invention, without these having been explicitly mentioned for reasons of clarity.

In the process of the invention for preparing linear octatrienes from 1,3-butadiene or 1,3-butadiene-containing hydrocarbon mixtures, the dimerization of the 1,3-butadiene is carried out in the presence of a secondary alcohol and a base and a complex of a metal of transition group eight of the Periodic Table of the Elements having at least one carbene of the structure L,

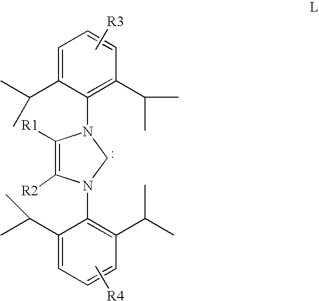

where R1 and R2=C₁-C₃-alkyl radical and R3 and R4=H or a C₁-C₃-alkyl radical, with the radicals R1 and R2 or R3 and R4 being able to be identical or different, as ligand is used as catalyst. The radicals R3 and R4 can be joined to the benzene ring in the 3, 4 or 5 position. The radicals R3 and R4 are preferably bound to the benzene ring in the 4 position. Particular preference is given to using complexes of the metals of transition group eight of the Periodic Table of the Elements having at least one 1,3-bis(2,6-diisopropylphenyl)-4,5-dimethyl-2-dehydro-3-hydroimidazole (structure L1) as ligand as catalysts for the dimerization of 1,3-butadiene to form unbranched acyclic octratrienes in the process of the invention.

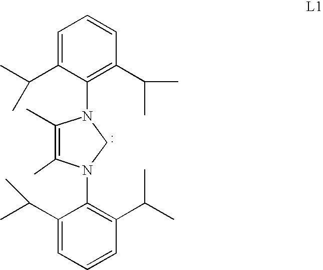

The (complex) catalysts used in the process of the invention can have one or more of the metals of transition group VIII of the Periodic Table of the Elements as catalyst metal. The catalysts used according to the invention preferably have nickel or palladium, particularly preferably palladium, as metal.

It can be advantageous for the dimerization of the 1,3-butadiene to form linear octratrienes to be carried out in the presence of at least one further ligand. Here, it is advantageous to use ligands which increase the reaction rate, improve the selectivity to the formation of linear octatrienes, increase the operating life of the catalyst or bring about other advantages as such additional ligands. The process of the invention for the dimerization of 1,3-butadiene can be carried out particularly advantageously when at least 1,1,3,3-tetramethyl-1,3-divinyldisiloxane (DVDS) is present as further ligand in addition to the carbene ligand L. The ratio of the optional further ligands, in particular DVDS, to the carbene ligand L is preferably from 0.1:1 to 10:1, more preferably from 0.5:1 to 1.5:1, particularly preferably from 0.9:1 to 1.1:1 and very particularly preferably 1:1.

In the carbene complexes of the invention, the metal, in particular palladium, is preferably present in the oxidation states 0 and 2. Examples of such carbene complexes are, inter alia, palladium(0)-carbene-olefin complexes, palladium-carbene-phosphine complexes, palladium(0)-dicarbene complexes, palladium(2)-dicarbene complexes, palladium(0)-carbene-diene complexes, palladium(2)-carbene-diene complexes and palladium(0)-carbene L-DVDS complexes.

The carbene complexes used in the process of the invention can be prepared in various ways. A simple route is, for example, the addition of the carbene L onto a metal compound, in particular a palladium compound, or the replacement of a ligand of a metal complex by the carbene of the structure L.

As precursors for the catalysts, it is possible to use, in particular, metal salts, preferably salts of organic acids or hydrohalic acids. Precursors which can be used for the palladium-containing catalysts are palladium salts, for example palladium(II) acetate, palladium(II) chloride, palladium(II) bromide, lithium tetrachloropalladate, palladium(II) acetylacetonate, palladium(0)-dibenzylideneacetone complexes, palladium(II) propionate, bisaceto-nitrilepalladium(II) chloride, bistriphenylphosphanepalladium(II) dichloride, bis-benzonitrilepalladium(II) chloride, bis(tri-o-tolylphosphine)palladium(0) and further palladium(0) and palladium(II) complexes.

Precursors which can be used for the nickel-containing catalysts are nickel compounds, for example [Ni(1,5-C₈H₁₂)₂], (c-C₅H₅)₂Ni, (Ph₂P(CH₂)₃PPh₂)₂NiCl₂, (PPh₃)₂NiBr, PPh₃Ni(CO)₂, nickel(II) acetylacetonate, nickel(II) chloride or similar nickel compounds listed in catalogs of chemical suppliers.

Precursors which can be used for the palladium-comprising catalysts are palladium compounds, for example bistriphenylphosphanepalladium(II) dichloride, bisbenzo-nitrilepalladium(II) chloride, bis(tri-o-tolylphosphine)palladium(0) and further palladium(0) and palladium(II) complexes.

The carbene L can be used as such or as metal complex or else be generated in situ from a precursor. The carbene of the structure L and the metal complex derived therefrom can be generated in situ from an imidazolium salt of the general structure S

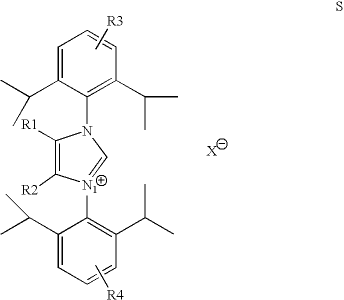

where R1 and R2=C₁-C₃-alkyl radical and R3 and R4=H or a C₁-C₃-alkyl radical, with the radicals R1 and R2 or R3 and R4 being able to be identical or different, and a base, in particular a metal compound. The particularly preferred carbene 1,3-bis(2,6-diisopropylphenyl)-4,5-dimethyl-2-dehydro-3-hydroimidazole and the metal complex derived therefrom can, for example, be generated in situ from a 1,3-bis(2,6-diisopropylphenyl)-4,5-dimethyl-3-hydroimidazolium salt of the general structure S1

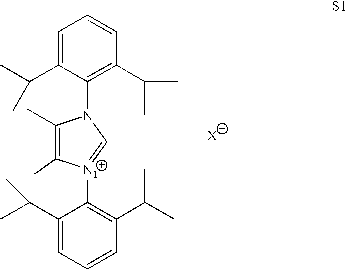

and an appropriate base, in particular an appropriate metal compound.

Examples of X⁻ are halides, hydrogensulfate, sulfate, sulfonates, alkylsulfates, arylsulfates, borates, hydrogencarbonate, carbonate, alkylcarboxylates, phosphates, phosphonates and arylcarboxylates. The carbene L or L1 is preferably set free from the salts of the structure S or S1 by reaction with a base. The precursors can be obtained in a known manner by reaction of appropriately substituted anilines with appropriately substituted 2,3-butanedione and formaldehyde. The preparation of such precursors is described, for example, in “Nucleophilic Carbenes and their Applications in modern Complex Catalysis, Anthony J. Arduengo and Thomas Bannenberg, The Strem Chemiker, June 2002, Vol. XVIV No. 1”.

If the catalyst is produced in situ from a palladium compound and a carbene precursor of the structure S, an alkoxide of the secondary alcohol used in the dimerization of the 1,3-butadiene is advantageously used as base. If, for example, isopropanol is used as solvent, it is advantageous to use isopropoxides as base. Preference is given to using alkali metal alkoxides, in particular sodium alkoxides. If desired, solutions comprising alkali metal hydroxide and alcohol can also be used in place of the alkoxide solutions.

In the process of the invention, the concentration of the catalyst, formally reported in ppm (mass) of metal based on the total mass, is preferably from 0.01 ppm to 1000 ppm, more preferably from 0.5 to 100 ppm and particularly preferably from 1 to 50 ppm. The ratio (mol/mol) of carbene L to metal can be from 0.01/1 to 250/1, preferably from 1/1 to 100/1 and particularly preferably from 1/1 to 50/1.

In the process of the invention, the 1,3-butadiene dimerization is preferably carried out in the presence of a secondary alcohol having from 3 to 20 carbon atoms. The alcohol used can be alicyclic or aliphatic. Preference is given to using secondary aliphatic alcohols, in particular linear alcohols. It is also possible to use mixtures of two or more alcohols. Furthermore, polyhydric, secondary alcohols, for example diols such as 2,4-dihydroxypentane, triols, tetraols etc., can also be used. Preferred alcohols are isopropanol and cyclohexanol. Particular preference is given to using isopropanol in the process of the invention.

The mass ratio of alcohol to 1,3-butadiene can be in the range from 1/20 to 20/1, preferably in the range from 4/1 to 1/2. Since the reaction should occur in a homogeneous liquid phase, these ranges are subject to restrictions only when 1,3-butadiene or the 1,3-butadiene-containing hydrocarbon mixture used has a miscibility gap with the alcohol.

The reaction mixture can optionally comprise further solvents, for example a high boiler in which the catalyst and possibly the base used dissolve(s).

According to the invention, the dimerization of the 1,3-butadiene to form linear octatrienes occurs in the presence of free base (base which is not used for generation of the carbene L). Preference is given to using alkoxides, particularly preferably alkoxides of the alcohol used, as base. Particular preference is given to using alkali metal alkoxides, in particular sodium alkoxide, as base. However, it is also possible to use alkali metal hydroxides such as NaOH or KOH as bases in the process of the invention.

The ratio of base to 1,3-butadiene is preferably from 0.01 mol to 10 mol per 100 mol of 1,3-butadiene, in particular from 0.1 mol to 5 mol and very particularly preferably from 0.2 mol to 1 mol per 100 mol of 1,3-butadiene.

The temperature at which the dimerization of the 1,3-butadiene to form linear octatrienes can be carried out is preferably from 10 to 180° C., more preferably from 40 to 100° C. and particularly preferably 40 to 80° C. The reaction pressure is preferably from 0.1 to 30 MPa, more preferably from 0.1 to 12 MPa, particularly preferably from 0.1 to 6.4 MPa and very particularly preferably from 0.1 to 2 MPa.

The dimerization of the 1,3-butadiene can be carried out continuously or batchwise and is not restricted to the use of particular types of reactor. Examples of reactors in which the dimerization can be carried out are stirred vessels, cascades of stirred vessels, flow tubes and loop reactors. Combinations of various reactors are also possible, for example a stirred vessel with a downstream flow tube.

The dimerization can, in order to obtain a high space-time yield, be carried out only to incomplete conversion of the 1,3-butadiene. It is advantageous to limit the conversion to not more than 95%, preferably not more than 90%.

The starting material for the process of the invention can be pure 1,3-butadiene or 1,3-butadiene-containing hydrocarbon streams, preferably 1,3-butadiene-rich hydrocarbon streams. In particular, a butadiene-containing C₄ fraction can be used as starting material. Apart from the 1,3-butadiene, the hydrocarbon streams used can comprise, inter alia, allenically unsaturated compounds. Particular preference is given to using a C₄-hydrocarbon fraction as hydrocarbon stream. The hydrocarbon streams are preferably, for example, mixtures of 1,3-butadiene with other C₄- and C₃- or C₅-hydrocarbons. Such mixtures are obtained, for example, in cracking processes for the production of ethylene and propylene in which refinery gases, naphtha, gas oil, LPG (liquified petroleum gas), NGL (natural gas liquid), etc., are reacted. The C₄ fractions obtained as by-product in the processes can comprise 1,3-butadiene together with monoolefins (1-butene, cis-but-2-ene, trans-but-2-ene, isobutene), saturated hydrocarbons (n-butane, isobutane), acetylenically unsaturated compounds (ethylacetylene, vinylacetylene, methylacetylene (propyne)) and also allenically unsaturated compounds (mainly 1,2-butadiene). In addition, these fractions can contain small amounts of C₃- and C₅-hydrocarbons. The composition of the C₄ fractions depends on the respective cracking process, the production parameters and the starting material. The concentrations of the individual components are typically in the following ranges:

| Component | % by mass |
|---|---|
| 1,3-Butadiene | 25-70 |
| 1-Butene | 9-25 |
| 2-Butenes | 4-20 |
| Isobutene | 10-35 |
| n-Butane | 0.5-8 |
| Isobutane | 0.5-6 |
| Σ Acetylenic compounds | 0.05-4 |
| 1,2-Butadiene | 0.05-2 |

In the process of the invention, preference is given to using hydrocarbon mixtures having a 1,3-butadiene content of greater than 35% by mass.

The starting hydrocarbons can frequently contain traces of oxygen compounds, nitrogen compounds, sulfur compounds, halogen compounds, in particular chlorine compounds, and heavy metal compounds which could interfere in the process of the invention. It is therefore advantageous to separate off these substances at the beginning. Interfering compounds can be, for example, stabilizers, tert-butylcatechol (TBC), or carbon dioxide or carbonyl compounds, e.g. acetone or acetaldehyde.

These impurities can be separated off by, for example, scrubbing, in particular with water or aqueous solutions, or by means of adsorbents.

A water scrub can completely or partly remove hydrophilic components, for example nitrogen components, from the hydrocarbon mixture. Examples of nitrogen components are acetonitrile or N-methylpyrrolidone (NMP). Oxygen compounds, too, can in part be removed by means of a water scrub. The water scrub can be carried out directly using water or else using aqueous solutions which may comprise, for example, salts such as NaHSO₃ (U.S. Pat. No. 3,682,779, U.S. Pat. No. 3,308,201, U.S. Pat. No. 4,125,568, U.S. Pat. No. 3,336,414 or U.S. Pat. No. 5,122,236).

It can be advantageous for the hydrocarbon mixture to go through a drying step after the water scrub. Drying can be carried out by methods known from the prior art. If dissolved water is present, drying can be carried out using, for example, molecular sieves as desiccant or by means of azeotropic distillation. Free water can, for example, be separated off by phase separation, e.g. using a coalescer.

Adsorbents can be used to remove impurities in the trace range. This can, for example, be advantageous because noble metal catalysts which react even to traces of impurities with a significant decrease in the activity are used in the second process step. Nitrogen compounds or sulfur compounds and also TBC are often removed by means of upstream adsorbents. Examples of adsorbents are aluminum oxides, molecular sieves, zeolites, activated carbon or metal-impregnated aluminas (e.g. U.S. Pat. No. 4,571,445 or WO 02/53685). Adsorbents are marketed by various companies, for example by Alcoa under the name Selexsorb®, by UOP or by Axens, e.g. the product series SAS, MS, AA, TG, TGS or CMG.

If the hydrocarbon stream used contains an amount of more than 100 ppm by mass of acetylenically unsaturated compounds, it can be advantageous in the process of the invention for the acetylenically unsaturated compounds to be separated off or removed from the hydrocarbon stream, which may have been purified beforehand, to a content of less than or equal to 100 ppm by mass, preferably less than or equal to 50 ppm by mass and particularly preferably less than or equal to 20 ppm by mass, in a preceding step before the hydrocarbon stream is used in the dimerization step. The separation/removal can be carried out, for example, by extraction or hydrogenation of the acetylenically unsaturated compounds. Any methylacetylene present can also be removed by distillation.

The removal of acetylenic compounds by extraction has been known for a long time and is, as work-up step, an integral part of most plants which isolate 1,3-butadiene from C₄ fractions from a cracker. A process for the removal of acetylenically unsaturated compounds from C₄ fractions from a cracker by extraction is described, for example, in Erdöl und Kohle-Erdgas-Petrochemie vereinigt mit Brennstoffchemie vol. 34, number 8, August 1981, pages 343-346. In this process, the multiply unsaturated hydrocarbons and the acetylenically unsaturated compounds are separated off from the monoolefins and saturated hydrocarbons by extractive distillation with water-containing N-methylpyrrolidone (NMP) in a first step. The unsaturated hydrocarbons are separated off from the NMP extract by distillation and the acetylenically unsaturated compounds having four carbon atoms are separated off from the hydrocarbon distillate by means of a second extractive distillation with water-containing NMP. In the work-up of C₄ fractions from a cracker, pure 1,3-butadiene is separated off by means of two further distillations, with methylacetylene and 1,2-butadiene being obtained as by-products. In the context of the process of the invention, the 1,3-butadiene obtained in this way can be the starting material for the dimerization.

The 1,3-butadiene-containing hydrocarbon streams obtained by extraction, which may further comprise 1,2-butadiene and/or less than 100 ppm by mass of acetylenic compounds, can be used either directly or after a work-up, preferably directly, as starting material in the dimerization.

The removal of acetylenically unsaturated compounds from the hydrocarbon stream used is preferably carried out by hydrogenation of the acetylenically unsaturated compounds. To avoid yield losses, especially of 1,3-butadiene, the hydrogenation process has to be very selective, i.e. the hydrogenation of 1,3-butadiene to linear butenes and the hydrogenation of butenes to butanes has to be very largely avoided. The selective hydrogenation of acetylenic compounds in the presence of dienes and monoolefins can be carried out using, for example, copper-containing catalysts. It is likewise possible to use catalysts comprising a noble metal of group VIII of the Periodic Table of the Elements, in particular palladium, or mixed catalysts. Particular preference is given to using copper-containing catalysts or catalysts comprising both palladium and copper.

As an alternative to hydrogenation, acetylenic compounds can also be removed from the butadiene-containing starting materials by reaction with an alcohol. Such processes are described, for example, in U.S. Pat. No. 4,393,249 and DD 127 082. Here, it can be advantageous for the alcohol used in the removal of the acetylenic compounds to be the same alcohol as used in the dimerization.

The reaction product mixture from the dimerization according to the invention can comprise, for example, linear octatrienes as main constituents and also by-products, “inert C₄-hydrocarbons”, residual amounts of 1,3-butadiene, secondary alcohol and catalyst system (metal complex, ligands, bases, etc.) or subsequent products thereof and any added solvent. Depending on the starting material mixture, 1,2-butadiene can also be present in the reaction product mixture. Any allenes present in the product mixture from the dimerization, in particular 1,2-butadiene, can be separated off by distillation.

The fractionation of the product mixture from the dimerization according to the invention can be carried out quite generally by means of known industrial processes, for example distillation or extraction. For example, a fractional distillation can be carried out to give the following fractions:

a C₄ fraction comprising n-butane, isobutane, 1-butene, 2-butenes, isobutene, 1,3-butadiene,
1,2-butadiene and possibly part of the alcohol,
a fraction comprising the linear octatrienes,
a fraction comprising the alcohol,
a fraction comprising by-products,
a fraction comprising the catalyst,
if appropriate a solvent fraction.

The fraction comprising the alcohol, the fraction comprising the solvent and the fraction comprising the catalyst or catalyst system can in each case be completely or partly recirculated to the dimerization or passed to a work-up.

The target product, i.e. the mixture of (linear) octatrienes prepared by the process of the invention, preferably comprises mainly 1,3,7-octatriene, i.e. at least 90% by mass of 1,3,7-octatriene. The two isomers (cis and trans) of 1,3,7-octatriene can be present in a ratio of, for example, about 1/1.7. The octratriene obtained can be used for the preparation of the products mentioned in the introduction, in particular for the preparation of linear octenes. In addition, the octatriene can be reacted with dienophiles to form the corresponding Diels-Alder products which can be utilized for modifying polyolefins or polyesters. Furthermore, 1,3,7-octatriene can be used as an intermediate for the preparation of 1-octene, which can be carried out using a method analogous to that for the preparation of 1-octene from 1,3,6-octatriene. Starting from 1,3,6-octatriene, 1-octene is prepared via the following route:
formation of a Diels-Alder adduct of anthracene and 1,3,6-octatriene, with the terminal double bond of the octatriene reacting,
hydrogenation of the two double bonds in the side chain of the adduct,
dissociation of the hydrogenated adduct (retro-Diels-Alder reaction) to give anthracene and 1-octene.

This synthetic route, which is described in Research Disclosures: “Process for preparing 1-Octene from Butadiene”, number 476, December 2003, page 1281, enables 1-octene to be prepared in a simple manner from butadiene via the octatrienes.

Furthermore, the linear octatriene prepared according to the invention can serve as precursor for the preparation of linear octenes, which can be prepared by selective hydrogenation. These are in turn valuable intermediates for the preparation of nonanols having a low degree of branching (by means of hydroformylation and hydrogenation), which can be utilized, inter alia, as plasticizer alcohols.

The C₄ fraction separated off from the reaction product mixture can be worked up in various ways. One way is firstly to separate the 1,2-butadiene from the C₄ fraction, e.g. by distillation, and pass it to a further use. Another way is to subject the C₄ fraction to a selective hydrogenation in which the dienes are removed, i.e. residual 1,3-butadiene and the 1,2-butadiene are converted into 1-butene and 2-butenes. Such hydrogenations are known from the prior art and are described, for example, in U.S. Pat. No. 5,475,173, DE 3119850 and F. Nierlich, F. Obenhaus, Erdöl & Kohle, Erdgas, Petrochemie (1986) 39, 73-78. In industry, they are carried out both in one stage and in a plurality of stages. The hydrogenation is preferably carried out in the liquid phase over heterogeneous supported palladium catalysts. Any alcohol present in the C₄ fraction can, if necessary, be separated off by known methods either before or after the hydrogenation. Readily water-soluble alcohols (for example isopropanol) can be removed, for example, by means of a water scrub. Drying columns, inter alia, have been found to be useful for drying the C₄ stream. The resulting mixture of largely 1,3-butadiene-, 1,2-butadiene- and alcohol-free C₄ hydrocarbons (butadiene content preferably less than 5000 ppm) corresponds largely to commercial raffinate I and can be processed further or worked up like raffinate I using known methods. For example, it can be used for the preparation of tert-butyl alcohol, diisobutene (or isooctane), methyl tert-butyl ether, 1-butene or C₄ dimers and oligomers.

The following example illustrates the invention without restricting its scope which is defined by the description and the claims.

### EXAMPLE 1

0.005 mol of a 1,3-bis(2,6-diisopropylphenyl)-4,5-dimethyl-3H-imidazolidenylpalladium(0)-DVDS-complex (complex formed by palladium, the ligand L1 and DVDS as further ligand) was dissolved in 33.3 g of a 0.5 molar solution of sodium isopropoxide in isopropanol. The mixture was introduced under argon into a 100 ml stainless steel Parr autoclave. The autoclave was cooled to −15° C. by means of a cooling bath. 15.0 g (2.77/10⁻¹ mol) of 1,3-butadiene from a pressure cylinder were subsequently condensed into the autoclave under mass control. After closing the autoclave, it was heated to 70° C. and the temperature was kept constant for 16 hours. The autoclave was then cooled to room temperature. The butadiene which left the autoclave on depressurization was condensed and weighed. 5 g of isooctane were added as internal standard to the remaining contents of the autoclave. The yield of linear octatriene was determined by gas chromatography using an HP 6869 A instrument. The yield of linear 1,3,7-octatrienes was 92% at a chemoselectivity of 95%. The chemoselectivity is the yield of octatrienes multiplied by 100 and divided by the sum of the yields of telomer, octatrienes and 4-vinylcyclohexene.

### EXAMPLE 2

Example 2 was carried out in a manner analogous to Example 1 except that the secondary alcohol was cyclohexanol and the base was sodium cyclohexoxide. The yield of linear 1,3,7-octatrienes was 83% at a chemoselectivity of 95%.

### EXAMPLE 3

Example 3 was carried out in a manner analogous to Example 1 except that 1,3-bis(2,6-diisopropylphenyl)-4,5-dimethyl-3-hydroimidazolium bromide and 0.005 mol % of palladium acetate (1.38*10⁻⁵ mol) in a ratio of 4:1 were used in place of the complex. The yield of linear 1,3,7-octatrienes was 83% at a chemoselectivity of 93%.

1. A process for preparing linear octatrienes from 1,3-butadiene or 1,3-butadiene-containing hydrocarbon mixtures, wherein the dimerization of the 1,3-butadiene is carried out in the presence of a secondary alcohol and a base, and a complex of a metal of transition group eight of the Periodic Table of the Elements having at least one carbene of the structure L 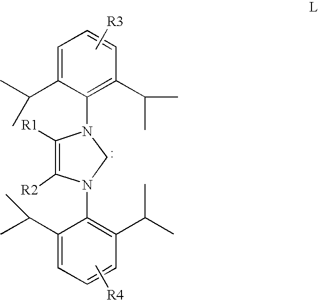
where R1 and R2=C₁-C₃-alkyl radical and R3 and R4=H or C₁-C₃-alkyl radical, with the radicals R1 and R2 or R3 and R4 being able to be identical or different, as ligand is used as catalyst.

2. The process as claimed in claim 1, wherein the catalyst comprises 1,3-bis(2,6-diisopropylphenyl)-4,5-dimethyl-2-dehydro-3-hydroimidazole (structural formula L1) as carbene ligand.

3. The process as claimed in claim 1, wherein a catalyst having palladium or nickel as metal is used.

4. The process as claimed in claim 1, wherein alkoxides are used as base.

5. The process as claimed in claim 1, wherein alkali metal hydroxides are used as bases.

6. The process as claimed in claim 1, wherein isopropanol is used as secondary alcohol.

7. The process as claimed in claim 1, wherein cyclohexanol is used as secondary alcohol.

8. The process as claimed in claim 1, wherein the carbene of the structure L and the metal complex derived therefrom are generated in situ from an imidazolium salt of the general structure S 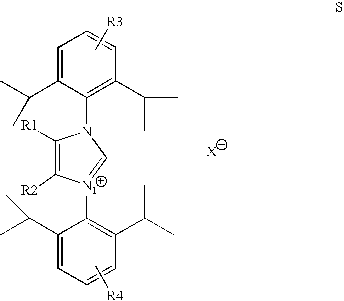
where R1 and R2=C₁-C₃-alkyl radical and R3 and R4=H or C₁-C₃-alkyl radical, with the radicals R1 and R2 or R3 and R4 being able to be identical or different, and a metal compound.

9. The process as claimed in claim 8, wherein the carbene 1,3-bis(2,6-diisopropylphenyl)-4,5-dimethyl-2-dehydro-3-hydroimidazole and the metal complex derived therefrom are generated in situ from a 1,3-bis(2,6-diisopropylphenyl)-4,5-dimethyl-3-hydroimidazolium salt of the general structure S1 and a metal compound.

10. The process as claimed in claim 1, wherein a butadiene-containing C₄ fraction is used as starting material.

11. The process as claimed in claim 1, wherein the concentration of the catalyst, formally reported in ppm (mass) of metal based on the total mass, is from 0.01 ppm to 1000 ppm.

12. The process as claimed in claim 1, wherein the ratio (mol/mol) of carbene L to metal is set in the range from 0.01/1 to 250/1.

13. The process as claimed in claim 1, wherein at least one further ligand is present in addition to the carbene ligand.

14. The process as claimed in claim 13, wherein at least 1,1,3,3-tetramethyl-1,3-divinyldisiloxane is present as a further ligand in addition to the carbene ligand.

15. The process as claimed in claim 13, wherein the ratio of the at least one further ligand to the carbene ligand L is preferably from 0.1:1 to 10:1.

16. A mixture of octatrienes prepared by the process as claimed in claim 1.

17. (canceled)

18. A carbene complex catalyst which is a ligand complex of a metal of transition group eight of the Periodic Table of the Elements which has at least one carbene of the structure L, 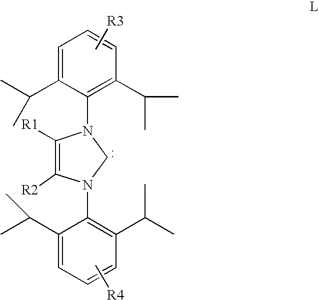
where R1 and R2=C₁-C₃-alkyl radical and R3 and R4=H or C₁-C₃-alkyl radical, with the radicals R1 and R2 or R3 and R4 being able to be identical or different, as ligand.

19. A process for preparing linear octenes comprising hydrogenating the mixture of octatrienes as claimed in claim 16.

20. Linear octenes produced by a process comprising hydrogenating the mixture of octatrienes as claimed in claim 16.
